(19) United States
(12) Patent Application Publication
(10) Pub. No.: US 2021/0154596 A1
(43) Pub. Date: May 27, 2021

(54) Cannabis Plant Extracts with Butane

(71) Applicant: Jenny's Rose, LLC, Los Angeles, CA (US)

(72) Inventor: James Castillo, San Diego, CA (US)

(21) Appl. No.: 16/691,132

(22) Filed: Nov. 21, 2019

(51) Int. Cl. B01D 11/02

(52) U.S. Cl. CPC B01D 11/0288; B01D 11/0219; C07D 311/80; B01D 11/0296; B01D 2201/184; B01D 11/028

(57) ABSTRACT

The present invention is a process for producing various types of cannabis extract from harvested cannabis. A quantity of harvested cannabis, which typically includes the inflorescence, floral leaves, and small stems of a flowering cannabis plants, is pre-frozen and is first subjected to cryogenic grinding to produce pulverized cannabis. The pulverized cannabis is then subjected to butane extraction at a low temperature to produce an initial cannabis extract. The butane in the initial cannabis extract is removed by evaporating it from the mixture in a cold vacuum chamber. The process preserves desirable waxes and lipids and removes the butane from the extract without subjecting the extract to heating in order to preserve the integrity of the terpenoids and flavonoids in the extract.

### U.S. GOVERNMENT SUPPORT

Not applicable.

### BACKGROUND OF THE INVENTION

The cannabis plant (Cannabis sativa L.) (“cannabis”) is an Asian, annual herbaceous plant that has been cultivated since antiquity for fiber, oil and food as well as for medicines. During this long history of cultivation, a number of different varieties have been selected. Tall, unbranched plants with large stems have been selected for fiber production; such plants are generally known as “hemp.” Shorter, usually heavily branched plants have been selected for their recreational and medicinal properties. Cannabis plants contain cannabinoids, a family of prenylated acylphloroglucinol derivatives that consist of over 100 different distinct chemical entities. Cannabinoids have a variety of different chemical structures, and the modern definition of “cannabinoid” is functional rather than structural: namely, they all are presumed to bind to cannabinoid receptors that are present on a variety of human cells. The cannabinoid receptors are part of a cellular signaling system known as the endocannabinoid system (ECS) and compounds that interact with these receptors can have profound physiological effects including effects on the central nervous system as well as the immune system.

In addition to cannabinoids, the cannabis plant also contains myriad other natural products such as terpenes and other terpenoids, and flavonoids that alter the taste, smell and physiological effect of cannabis. Although terpenoids and flavonoids are known to have potential medical and recreational benefits, medical as well as recreational effects of cannabis have been generally attributed to cannabinoids. The story is even more complicated because it is becoming recognized that cannabinoids interact with each other as well as with terpenoids and possibly other compounds present in the plant to produce medical and recreational effects that cannot be attributed to cannabinoids alone. This interaction or synergy resulting from the complex mixture of compounds present within the cannabis plant is known as the “entourage effect,” in which a mixture of compounds from the cannabis plant is believed to demonstrate greater efficacy in treating a medical condition than any of its constituent compounds in isolation.

Historically, cannabis has been consumed by smoking or ingestion. Cannabis resin known as ‘hashish’ was a well-known product in many countries, particularly in Asia. Smoking herbal material is not always convenient, and for oral ingestion (“edibles”) or medicinal preparations, unprocessed plant material containing unwanted components is often not ideal. Therefore, improved technology for producing cannabis extracts was developed. These concentrates were primarily extracts of cannabinoids produced by using organic solvents to dissolve cannabinoids and other natural products from cannabis plant material. Initially, these extracts were rather crude and contained a wide variety of natural products (such as chlorophyll and plant waxes) and contaminants present in the plant materials, as well residual organic solvents. As the demand for cannabis extracts has increased, improved technologies for producing high quality cannabis extracts are being developed.

Organic solvents such as butane, that are gases at room temperature, were adopted for the extraction process because these solvents can be readily removed from the extract by mild heating at atmospheric pressures. It will be appreciated that butane gas can be explosive so that complex equipment is required for butane extraction processes. Liquid carbon dioxide was also adopted as a non-flammable solvent, but as might be expected the equipment to use carbon dioxide as an extracting solvent is also expensive and complex. It will also be appreciated that each different solvent may extract a somewhat different range of plant compounds in addition to cannabinoids. Cannabis extracts were originally developed on a hit or miss basis. Although cannabis plants contain many different cannabinoids as well as a variety of other bio-active compounds, most early extracts were tested only to see if the extract made one “high” following consumption of the extract. This type of “testing” naturally favored processes that efficiently extracted the psychoactive cannabis compounds—little consideration was given to other bioactive, but not psychoactive, cannabis compounds.

In performing a cannabis-butane extraction, after an initial soaking of the plant materials in the butane to extract the compounds soluble in butane, subsequent processing steps were typically executed to remove unwanted materials such as chlorophyll, waxes, and lipids from a crude cannabis extract because such elements were viewed to interfere with the effectiveness and stability of the product and were not desirable in the final product. This required further purification which involves the discarding of fractions containing the undesirable compounds. Thus, in the typical extraction process, the goal is to obtain extracts containing high concentrations of the cannabinoids, while discarding portions of the extract containing waxes, lipids, and other components of plant materials.

Surprisingly, it has been found that the fraction that is typically discarded, probably containing waxes and lipids, is important to the therapeutic value of the extract and contributes to the entourage effect of the extract. Capturing the portions of the extract that would preserve the “entourage effect,” while removing unwanted plant matter from the extraction process is challenging. When breaking down the plant matter, the initial extract may contain particulate materials, chlorophyll, large sugars, such as plant cellulose, and proteins that are not beneficial and affect other aspects of the product, such as its shelf-life and stability. Thus, there is a need to develop an extraction process whereby unwanted plant materials are removed, but desirable waxes and lipids, which add to the therapeutic value of the extract, are preserved.

The initial extract must be further processed to remove the solvent and optionally, to decarboxylate the cannabinoids. The solvent must be removed as it is an unwanted contaminant. Decarboxylation is required to convert the naturally occurring acidic forms of the cannabinoids into the physiologically active forms. For example, the psychoactive compounds Δ9-THC (Δ9-tetrahydrocannabinol) and THCV (tetrahydrocannabivarin) are naturally present in the plant as inactive THCA (tetrahydrocannabinolic acid) and THCVA (tetrahydrocannabivarinic acid), and must be decarboxylated into the biologically active forms. Decarboxylation can be accomplished in a variety of different ways, and is typically accomplished by heating the acidic form of the cannabinoids. In the butane extraction process, a commonly used method is to place the extract in a vacuum oven and heat it to between about 26° C. to about 140° C. At this temperature, the solvent evaporates and the cannabinoids are decarboxylated. However, a shortcoming of heating the extract to such high temperatures is that causes the loss of more volatile compounds, including terpenoids and flavonoids that otherwise add to the value to the extract.

Accordingly, there is a need for a butane extraction process that preserves the desirable plant waxes and lipids and removes the organic solvent from the extract without subjecting the extract to heating to high temperatures in an oven to preserve the integrity of volatile compounds including the terpenoids and flavonoids in the extract.

### SUMMARY OF THE INVENTION

In accordance with the invention, provided is a process for producing various types of cannabis extract from harvested cannabis. A quantity of harvested cannabis, which typically includes the inflorescence, floral leaves, and small stems of a flowering cannabis plants, is pre-frozen. The harvested cannabis is first subjected to cryogenic grinding to produce pulverized cannabis. The pulverized cannabis is then subjected to hydrocarbon extraction at a low temperature to produce an initial cannabis extract. The hydrocarbon in the initial cannabis extract is removed by evaporating the hydrocarbon from the mixture in a cold vacuum chamber.

In one embodiment, the invention provides a method of producing a cannabis extract and a “remainder” fraction (believed to comprise plant waxes, lipids and other active components) comprising the steps of: immersing a quantity of pulverized cannabis in solvent of liquefied hydrocarbon gas at a temperature between about −15° C. and about −35° C. to produce a solvent-rich cannabis extract; passing the solvent-rich cannabis extract through one or more filters to obtain an initial cannabis extract which passes through the one or more filters and a remainder fraction that is retained by the filters; and subjecting the initial cannabis extract to purging in a vacuum chamber at initial temperatures between about 0° C. and −35° C. to yield a cannabis extract essentially free of solvent.

In one embodiment, the hydrocarbon gas is chilled to between about 0° C. and about −70° C. In another embodiment, the hydrocarbon gas is chilled to between about −5° C. and about −60° C., or to between about −10° C. and about −50° C., or to between about −15° C. and about −40° C., or to between about −20° C. and about −35° C. or to any temperature in between.

In one embodiment, the temperature in the vacuum chamber is between about −70° C. to about 50° C. In another embodiment, the temperature in the vacuum chamber is between about −60° C. to about 40° C., or between about −50° C. to about 30° C., or between about −40° C. to about 30° C., or between about −30° C. to about 20° C., or between about −20° C. to about 10° C., or between about −10° C. to about 0° C., or any temperature in between.

### DESCRIPTION OF THE FIGURES

FIG. 1 illustrates an extraction device.

FIG. 2 illustrates the bi-directional column with an arrangement of filters at both ends of the column.

### DETAILED DESCRIPTION OF THE INVENTION

The following description is provided to enable any person skilled in the art to make and use the invention and sets forth the best modes contemplated by the inventor of carrying out his invention. Various modifications, however, will remain readily apparent to those skilled in the art, since the general principles of the present invention have been defined herein specifically to provide a method for extraction of cannabinoids using butane.

Embodiments of the invention are discussed in detail below. In describing embodiments of the invention, specific terminology is employed for the sake of clarity. However, the invention is not intended to be limited to the specific terminology so selected. While specific exemplary embodiments are discussed, it should be understood that this is done for illustration purposes only. A person skilled in the relevant art will recognize that other components and configurations can be used without parting from the spirit and scope of the invention. All references cited herein are incorporated by reference as if each had been individually incorporated.

The term “cannabis extract” is used to refer to cannabis extracts that may contain cannabinoids and other beneficial components, such as terpenoids, or their synthetic derivatives or functional equivalents. In one embodiment, cannabinoids may comprise any one of, or a mixture comprising, THC (tetrahydrocannabinol), THCA (tetrahydrocannabinolic acid), CBD (cannabidiol), CBDA (cannabidiolic acid), CBN (cannabinol), CBG (cannabigerol), CBC (cannabichromene), CBL (cannabicyclol), CBV (cannabivarin), THCV (tetrahydrocannabivarin), CBDV (cannabidivarin), CBCV (cannabichromevarin), CBGV (cannabigerovarin), CBGM (cannabigerol monomethyl ether), CBE (cannabielsoin), or CBT (cannabicitran), for example.

Terpenoids encompass a broad group of organic compounds that include terpenes, diterpenes, and sesquiterpenes. More than one hundred different terpenoids have been detected in cannabis. In one embodiment, cannabis extract may comprise terpenoids. In another embodiment, terpenoids may comprise alpha-bisabolol, borneol, alpha-caryophyllene, beta-caryophyllene, elemene (alpha, beta, gamma, or delta), limonene, camphene, camphor, delta-3-carene, caryophyllene oxide, alpha-cedreen, citral, eucalyptol, beta-eudesmol, eudesm-7(11)-en-4-ol, farnesene, fenchol, alpha-guaiene, geraniol, guaiol, germacrene B, guaia-1(10)-11-diene, humulene, alpha-humulene, isobomeol, linalool, menthol, myrcene, alpha-myrcene, beta-myrcene, nerol, cis-ocimene, trans-ocimene, alpha-phellandrene, alpha-pinene, beta-pinene, pulegone, sabinene, alpha-terpinene, alpha-terpineol, terpinolene, terpineol, thymol, trans-2-pinanol, selina-3,7(1)-diene, or valencene.

### Hydrocarbon Solvent

It will be appreciated that the hydrocarbon solvent is chosen so that the boiling point of the solvent is below normal ambient temperatures—that is, the solvent is a liquefied hydrocarbon gas. This ensures that the solvent can be removed without subjecting the cannabis extract to high temperatures. In one embodiment, butane is used as a hydrocarbon solvent for cannabinoid extraction. In other embodiments, the hydrocarbon solvent may be a single hydrocarbon solvent, such as n-butane, isobutane, or propane. In another embodiment, the hydrocarbon solvent may be a mixture of multiple hydrocarbon compounds. In still another embodiment, the hydrocarbon solvent may be a mixture of two hydrocarbon compounds at a ratio of 1:5, 1:4, 1:3, 1:2, 1:1, or any ratio in between.

In one embodiment, the hydrocarbon solvent may be a mixture of two hydrocarbon compounds. In other embodiments, the hydrocarbon solvent may be a 99%-1%, 90%-10%, 80%-20%, 70%-30%, 60%-40%, 55%-45%, or 50%-50% mixture of two hydrocarbon compounds, or a mixture at any percentage in between. In one embodiment, the hydrocarbon solvent may be a 60%-40% mixture of propane and n-butane. In another embodiment, the hydrocarbon solvent may be a 60%-40% mixture of n-butane and isobutane.

Cannabis plants grown outdoors are typically contaminated with dirt and insects, for example. Cannabis Extraction using butane as the solvent may lead to inclusion of high levels of impurities in the final cannabis extract. An extraction method that uses propane instead of butane with cannabis plants grown outdoors reduces the levels of impurities in the final cannabis extract. However, the cannabinoid yield is lower. For cannabis plants grown indoors, using n-butane/isobutane mixture as solvent can increase the quality of cannabis extracts.

### Butane Extraction Methods

Existing butane extraction methods are often conducted at ambient temperature on dry plant material. In the present extraction method plant material is preferably prepared by cryogenic grinding of frozen plant material as described in U.S. Pat. No. 10,471,113. If frozen plant material is cryogenically ground, butane extraction at ambient temperature can lead to thawing of the frozen plant material which can result in loss of flavonoids and terpenes with the resulting cannabis extract being deficient in volatile flavonoids, and terpenoids.

With the method of the present invention, butane extraction is conducted in a temperature controlled room. Extraction process is preferably carried out at an ambient room temperature below about 18.3° C. (65° F.). More preferably, the ambient room temperature is maintained at 10° C. (50° F.) or lower to reduce the risks of accidental ignition of the hydrocarbon solvent gas. In a preferred embodiment, harvested cannabis is cryogenically ground and kept frozen. Butane is chilled before contacting cryogenically ground cannabis plant matter. Preferably, butane is chilled to about −20 to −35° C. Cannabis plant matter is soaked in chilled butane in a bi-directional column, which is maintained at a temperature of about −25 to −30° C. Soaking cannabis plant matter in butane produces crude cannabis extract, which is then filtered.

Existing butane extraction methods also employ a step of removing, or “purging,” butane from the crude extract by placing the butane and cannabis extract mixture in a heated vacuum oven. Sustained exposure of cannabis extract to high temperatures can have detrimental effects, including the loss of volatile compounds such as flavonoids and terpenes through evaporation, thermal decomposition and uncontrolled decarboxylation of cannabinoids in the extract.

In the method of the invention, filtered cannabis extract is placed in a chilled vacuum chamber in order to remove butane. The novel method of using a chilled vacuum chamber, instead of an oven, reduces the loss of volatile terpenes and flavonoids. Further, using a chilled vacuum chamber prevents thermal decomposition and uncontrolled decarboxylation of cannabinoids in the extract.

### Filter Sizes

The size of filters are selected based on the desired final product. In one embodiment, filters with pore sizes including 220 μm, 100 μm, 45 μm, 32 μm, or 5 μm are used to filter the butane and cannabis extract mixture. It will be appreciated that a wide range of filter sizes can be employed. Initially, filters with relatively large pore sizes (e.g., 220 μm or 100 μm) are used to remove the majority plant material particles. Usually, a small pore size (e.g., 5 μm) is used to “polish” the extract and remove any very small particles.

### Remainder

In experimenting with various filter pore sizes, the inventor found that even when an initial “polishing” filtration was carried out using intermediate pore sizes (e.g., 30-50 μm), additional insoluble material could be removed with a small pore size filter. Insoluble material that is caught on the surface of the 5 μm filter following intermediate pore size filtration is known as “remainder” or “remainder fraction.” The remainder fraction can be recovered for use in therapeutic formulations. As detailed in co-pending U.S. patent application Ser. No. 16/569,535, filed on Sep. 12, 2019, the remainder fraction is believed to include micelles of plant waxes and other lipids, and very small particles of insoluble plant material. It has been unexpectedly found that adding remainder fraction to various medicinal or even recreational formulations intended for oral administration significantly augments the activity of these formulations. It is believed that some compounds within the remainder fraction interact with active cannabis compounds to produce an “entourage” effect. Alternately or additionally, lipids or other compounds in the remainder fraction enhance absorption of the formulation thereby increasing apparent activity. Because of the great utility of the remainder fraction, use of the 5 μm filtration step to recover the remainder fraction has become the preferred procedure. The remainder fraction, if used immediately on collection, may be added directly to cannabinoid extracts. If intended for later use, the remainder fraction may be suspended in ethanol, and stored at low temperatures for extended periods. Prior to use, the resuspended remainder may be recovered by centrifugation or filtration.

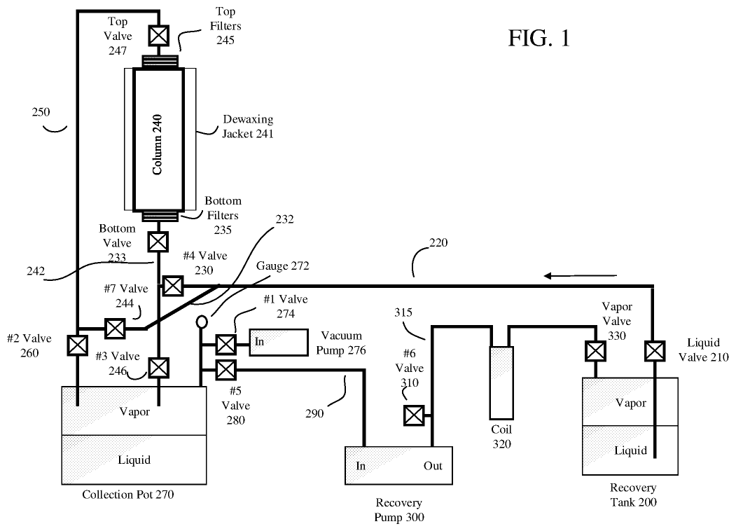

FIG. 1 is an illustration of one embodiment of an extraction device. As shown in FIG. 1, the extraction device may comprise: a Recovery Tank 200, a Liquid Valve 210, a first Hose 220, a #4 Valve 230, a second Hose 232, a #7 Valve 244, a Bottom Valve 233, a set of Bottom Filters 235, a Bi-directional Column 240, a set of Top Filters 245, a Top Valve 247, a #3 Valve 246, a third Hose 250, a #2 Valve 260, a Collection Pot 270, a Pressure Gauge 272, a #1 Valve 274, a Vacuum Pump 276, a #5 Valve 280, a fourth Hose 290, a Recovery Pump 300, a #6 Valve 310, a Vacuum Pump 311, a fifth Hose 315, a Coil 320, and a Vapor Valve 330. In a specific embodiment, the first Hose 220 connects the Recovery Tank 200 to the bottom of the Bi-directional Column 240 via the Liquid Valve 210, the #4 input Valve 230, and the Bottom Valve 233. The Recovery Tank 200 is also connected to the top of the Bi-directional Column 240 via the Liquid Valve 210, the second Hose 232, the #7 Valve 244, the third Hose 250, and the Top Valve 247. The third Hose 250 also connects the top of Column 240 to the Collection Pot 270 via the #2 Valve 260. The Hose 242 connects the bottom of the Column 240 to the Collection Pot 270 via the Bottom Valve 233 and the #3 Valve 246. The fourth Hose 290 connects the Collection Pot 270 to the Recovery Tank 200 via the #5 Valve 280, the Recovery Pump 300, the Coil 320, and the Vapor Valve 330. Bold arrows show direction of liquid flow and hashed arrows show direction of vapor flow.

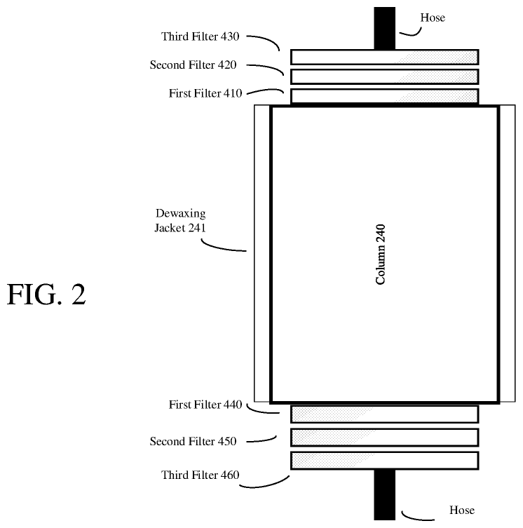

FIG. 2 is an illustration of one embodiment of the Bi-directional Column with an arrangement of filters at both ends of the column. The Bi-directional Column 240 has a dewaxing jacket 241. As shown in FIG. 2, one embodiment of the Bi-directional Column 240 may comprise: a first filter 410, 440 arranged such that it is closest to the column at each end of the column; a second filter 420, 450 placed next to the first filter; and a third filter 430, 460 placed next to the second filter. The filters may be 100 μm, 45 μm, 32 μm, or 5 μm in size.

### Extraction of Cannabinoids Using Butane

### Step 1

Butane (or other solvent) is distilled to remove any contaminants that may be present.

### Step 2

An MK4c Terpenator® with Bi-Directional Modification Kit, made by Terpp Extractors of Fort Collins, Colo., unit was used in the extraction process. Extraction may also be done using the Delta Technologies MK-420 extraction system made by Delta-9 Technologies of Lake Forest, Calif.

Bi-directional apparatus for extraction is set up. A schematic for extraction is illustrated in FIG. 1. The Hose 220 connects the Recovery Tank 200 to the bottom of the Bi-directional Column 240 via the Liquid Valve 210, the #4 Input Valve 230, and the Bottom Valve 233. The Recovery Tank 200 is also connected to the top of the Bi-directional Column 240 via the Liquid Valve 210, the Hose 232, the #7 Valve 244, the Hose 250, and the Top Valve 247. The Hose 250 also connects the top of the Column 240 to the Collection Pot 270 via the #2 Valve 260. The Hose 242 connects the bottom of the Column 240 to the Collection Pot 270 via the #3 Valve 246. The Hose 290 connects the Collection Pot 270 to the Recovery Tank 200 via the #5 Valve 280, the Recovery Pump 300, the Condensing Coil 320, and the Vapor Valve 330.

As shown in FIG. 1, two groups (235 and 245) of filters are added to the bottom and top ends of the column. The arrangement of the filters in the column is further illustrated in FIG. 2. The Bi-directional Column may comprise: a first filter 410, 440 arranged such that it is closest to the column at each end of the column; a second filter 420, 450 placed next to the first filter; and a third filter 430, 460 placed next to the second filter. The filters may be 100 μm, 45 μm, 32 μm, or 5 μm in size. The filters may be added to both ends of the column in order to prevent large particles of ground cannabis plant material from traveling out of the column and being deposited into the Collection Pot 270. Stacked filters are used to prevent formation of plugs in the filter and to maintain free flow of the butane and cannabis extract mixture out of the column and into the collection pot. A 5 μm filter is used in order to isolate “remainder” from the mixture.

### Step 3

A quantity of harvested cannabis may be prepared for extraction through cryogenic grinding. Cryogenic grinding physically breaks the harvested cannabis into very fine particles, which facilitates the dissolution of compounds present within the harvested cannabis by the hydrocarbon solvent. The cannabis plant material is kept frozen before, during, and after cryogenic grinding. A method of cryogenic grinding is described in U.S. patent application No. 16/365,614, which is incorporated herein in its entirety by reference.

### Step 4

To facilitate maintaining a low temperature within the extraction apparatus, the ambient temperature is maintained preferably below 18.3° C. (65° F.). More preferably, the ambient temperature is maintained at 10° C. (50° F.). Temperature of butane and the column is adjusted by means of the dewaxing jacket 241 which surrounds the Bi-directional Column 240. Cryogenic fluids are circulated through the jacket to adjust the column temperature which is selected depending on the desired product. The temperature at which the dewaxing process occurs may be between −15 to −35° C. depending on the desired product. For example, if a cannabis extract commonly known as “shatter,” which is consumed by smoking, is desired, the desired temperature of the column is closer to −35° C. This causes a larger amount of the waxes and lipids present in the extract to be deposited on the chilled outer surface of the column. Whereas, if the desired final product is intended to be used as a medicine or an edible, then the desired temperature is closer to −15° C. so that the “remainder” fraction can be collected by filtration. Further, if the desired final product is to be consumed orally, then the desired temperature is closer to −25° C.

The temperature of butane in Recovery Tank (140) is adjusted such that it is preferably 5° C. warmer than the column temperature. Preferably, the temperature of butane in the Recovery Tank (140) is adjusted to between −20 to −25° C. Whereas, the temperature of the Column 240 is adjusted between −15 to −35° C., or preferably to between −25 to −30° C.

### Step 5

An empty sock filter is placed inside the column and filled with cryogenically ground cannabis plant material. The 220 μm sock filter can be about 10 cm (4 inch) long. Cannabis plant material inside the sock filter is packed gently and sealed. The capacity of the sock filter may be up to 5 lbs (2.27 kg).

### Step 6

The Column 240 is sealed by closing the #4 Valve 230 and the #7 Valve 244. The Vacuum pump 276 is turned on to remove air from the Column 240 and the Collection Pot 270. Note that the Vacuum Pump 276 is moveable and can be attached to the #1 Valve 274 (as in the present case) to evacuate the Column 240 or to the #6 Valve 310 to evacuate other parts of the system. The vacuum pump is turned off once desired pressure, preferably 30 mmHg (about 4.0×10³ Pa), is reached. The #3 Valve, the #1 Valve, the #4 Valve, the #2 Valve, and the #7 Valve are then closed to isolate the column 240.

### Step 7

The column chamber is filled slowly from the bottom by opening the Bottom Valve 233 and the #4 Valve 230, allowing butane from the Recovery Tank 200 to fill the Column 240. Butane flows into the column from the Recovery Tank 200 through the Liquid Valve 210 and the #4 Valve 230.

### Step 8

Once the flow of butane from the Recovery Tank 200 into the column equilibrates, the #2 Valve 260 is opened to allow butane vapor to move into the Collection Pot 270. Once the line is flushed (i.e. butane vapor from the column and the hose 250 is flushed into the Collection Pot 270 and the first liquid butane starts to enter the collection pot), the #2 Valve 260 is closed. This ensures that liquid butane has filled the column 240, thus allowing cannabis plant material to be fully immersed in butane. Once the column is filled with butane, the #4 Valve 230 is closed.

### Step 9

Cannabis plant material is soaked in butane for 20-60 min depending on desired end product such as wax, shatter, or medication for therapeutic consumption.

Longer soak times produce a more cannabinoid-rich extract because of the increased time for cannabinoids to dissolve into the butane solvent. The Bi-directional Column 240 has a chilled dewaxing jacket 241, which allows inline dewaxing resulting from dissolved waxes and lipids precipitating on the chilled inner column surface. Longer soak times allow more of the plant waxes and other lipids to stick to the inner surface of the column, resulting in an extract that produces less of the “remainder” when filtered. As mentioned above, the process of cooling the mixture to a temperature at which a portion of waxes and lipid components precipitate is also known as “winterization.”

### Step 10

The Top Valve 247, the #2 Valve 260, the #5 Valve 280, and the Vapor Valve 330 are opened to allow the initial cannabis extract and butane to enter the Collection Pot 270 with suction provided by the Recovery Pump 300. The valves are kept open until the butane and cannabis extract mixture has completely transferred into the Collection Pot 270.

### Step 11

The Column 240 is sealed by closing the #4 Valve 230 and the #7 Valve 244. The Recovery pump 300 is turned on to remove gas from the column 240 and the Collection Pot 270. The Recovery pump 300 is turned off once desired pressure, preferably 30 mmHg (about 4.0×10³ Pa), is reached. The #3 Valve, the #1 Valve, the #4 Valve, the #2 Valve, and the #7 Valve are then closed to isolate the Column 240.

### Step 12

Column chamber is now filled slowly from the top by opening the #7 Valve 244, thereby allowing butane to fill the column. Butane flows into the column from the Recovery Tank 200 through the Liquid Valve 210 and the #7 Valve 244.

### Step 13

Once the flow of butane from the Recovery Tank 200 into the column equilibrates, The #3 Valve 246 is opened to allow butane vapor to move into the Collection Pot 270. Once the line is flushed (i.e. all of the butane vapor from the column and the hose 242 is flushed into the Collection Pot 270 and the first liquid butane beings to enter the Collection Pot 270, the #3 Valve 246 is closed. This ensures that liquid butane has filled the column 240, thus allowing cannabis plant material to be fully immersed in butane. Once the column is filled with butane, the #7 Valve 244 is closed.

### Step 14

Cannabis plant material is soaked for about 20-60 minutes to extract soluble material missed by the first extraction (Step 9) yielding a rinse extract.

Butane and cannabis rinse extract mixture is flushed through the bottom of the Column 240 by opening the #3 Valve 246. The #5 Valve 280 and the Vapor Valve 330 are opened to flush out the column. With the initial extraction (Step 9) the butane solution exits the top of the column (Step 10) and particulate material and remainder is captured on the top filters 245. When butane is returned to the column for the second extraction (Step 14) it enters the column from the top (Step 12) dislodging remainder and other material from the top filters 245. When the rinse extract is flushed out of the column through the bottom filters 235, this material collects on the bottom filters and the remainder is subsequently harvested from the Third Filter 460 (see FIG. 2).

### Step 15

Butane and cannabis extract mixture (both the initial extract and the rinse extract) is recovered from the Collection Pot 270. This leaves the cannabis oil (containing a small amount of residual butane) in the Collection Pot 270. The cannabis extract mixture is then poured onto parchment paper in a glass dish. In one embodiment, the glass dish is made from low-thermal-expansion glass, such as Pyrex® glass made by Corning Inc. of Tewksbury, Mass.

Traditionally, butane and cannabis extract mixture is placed into an oven set at about 37.8° C. (100° F.) or higher to remove residual butane from the cannabis extract. However, at this temperature at least some cannabinoids may be decarboxylated. This heating also results in the loss of some of the terpenes and terpenoids.

In contrast, the invention includes a novel method of removing butane from the mixture by requiring that the butane is evaporated from the butane and cannabis extract mixture in a cold vacuum chamber. The chamber is prepared by placing it in a cold room and the temperature of the chamber is lowered to between 0 to −35° C. by adding dry ice (solid carbon dioxide). Avoiding the traditional heating step allows more retention of volatile terpenoids as well as control of the decarboxylation process.

This process separates out cannabis extract from butane because neither the cannabinoids nor terpenoids are volatile under these temperature and pressure conditions while butane continues to evaporate at −31° F., particularly under reduced pressures. After placing the cannabis extract mixture in the chilled chamber, air is removed from the chamber by means of a vacuum pump that is left running for 3 to 5 hours. As the dry ice inside the chamber evaporates, the internal temperature of the chamber is allowed to increase to between about 24° C. and about 27° C. and the mixture is kept under vacuum for 48 hours to remove (“purge”) residual butane. After 48 hours the extract is removed, flipped over, and returned to the chamber until the purge is complete at between about 24° C. and about 27° C. The purging process may take between 96 hours to 168 hours to remove all traces of the butane. Processing the extract at a low temperature reduces the chances of uncontrolled decarboxylation of cannabinoids, or the loss of volatiles such as terpenoids, in the mixture. Once the purging is complete, the extract does not contain significant amounts of decarboxylated cannabinoids. The extract may then be decarboxylated through processes described in U.S. Pat. No. 10,471,113 or through other more traditional methods.

### Step 16

The Top Valve 247 and the Bottom valve 233 are closed allowing the Bottom filters 235 to be removed. The Remainder fraction is recovered from the surface of the filter with the smallest (5 μm) pore size and for use in formulations.

The following claims are thus to be understood to include what is specifically illustrated and described above, what is conceptually equivalent, what can be obviously substituted and also what essentially incorporates the essential idea of the invention. Those skilled in the art will appreciate that various adaptations and modifications of the just-described preferred embodiment can be configured without departing from the scope of the invention. The illustrated embodiment has been set forth only for the purposes of example and that should not be taken as limiting the invention. Therefore, it is to be understood that, within the scope of the appended claims, the invention may be practiced other than as specifically described herein.

What is claimed is:

1. A method of producing a cannabis extract and remainder fraction comprising the steps of:
immersing a quantity of pulverized cannabis in solvent of a liquefied hydrocarbon gas at a temperature between about −15° C. and about −35° C. to produce a solvent-rich cannabis extract;
passing the solvent-rich cannabis extract through one or more filters to obtain an initial cannabis extract which passes through the one or more filters and a remainder fraction that is retained by the filters;
purging the initial cannabis extract in a vacuum chamber at initial temperatures between about 0° C. and −35° C. to yield an essentially solvent-free cannabis extract.

2. The method of claim 1, wherein the liquefied hydrocarbon gas is selected from the group consisting of n-butane, isobutane, propane and mixtures of the same.

3. The method of claim 2, wherein the liquefied hydrocarbon gas is a mixture of n-butane and isobutane at a ratio of 6:4.

4. The method of claim 2, wherein the liquefied hydrocarbon gas is a mixture of propane and n-butane a ratio of 6:4.

5. The method of claim 1, wherein the filters have pore sizes between about 220 μm and about 5 μm.

6. The method of claim 5, wherein the remainder fraction is retained by the filter having a pore size of about 5 μm.

7. The method of claim 1, wherein the vacuum chamber is allowed to warm up to between about 24° C. and about 27° C.

8. The method of claim 1, wherein the step of purging takes between about 96 hours and about 168 hours.

9. The method of claim 1, wherein the initial temperature of the vacuum chamber is achieve by placing solid carbon dioxide into the vacuum chamber.

10. The method of claim 1, wherein the pulverized cannabis results from cryogenic grinding of frozen cannabis.

11. The method of claim 1, wherein the step of immersing takes place in a bidirectional column equipped with a chilled dewaxing jacket.

12. The method of claim 1, wherein the step of immersing lasts about 20 to about 60 minutes.
